(19) United States
(12) Patent Application Publication
(10) Pub. No.: US 2007/0020663 A1
(43) Pub. Date: Jan. 25, 2007

(54) Reaction monitoring systems

(76) Inventors: Peter Hagerlid, Uppsala (SE); Bjorn Ekstrom, Uppsala (SE); Jonas Sjoberg, Umea (SE)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/472,112

(22) Filed: Jun. 21, 2006

Related U.S. Application Data
(62) Division of application No. 09/719,960, filed on Jun. 14, 2001, now abandoned.

(30) Foreign Application Priority Data
Jun. 18, 1998 (GB) 9813216.0

(51) Int. Cl. C12Q 1/68; C12M 1/34

(52) U.S. Cl. 435/6; 435/287.2

(57) ABSTRACT

The present invention provides an apparatus for simultaneously monitoring an array of reaction sites for light indicating that a reaction is taking place at a particula site comprising a charge-coupled device (CCD) arranged so that in use the light from a particular reaction site will impinge upon a particular predetermined region of the CCD. The apparatus also has means for determining the light level impinging upon each of the predetermined regions and means to record the variation of light level with time for each of the reaction sites. Also disclosed is a method of identifying a target base in a DNA sequence using such an apparatus.

This invention relates to a method and apparatus for monitoring reactions and relates particularly, but not exclusively, to reactions which take place in DNA sequence determination.

There is a growing need today to be able to sequence efficiently large numbers of relatively short strands of DNA. A particularly useful method for doing this is the sequencing-by-synthesis method disclosed in WO 98/13523. In this method a complementary DNA strand is constructed using the normal rules of base pairings to allow the sequence of the fragment of interest to be determined. Successive deoxynucleotides are added cyclically, but only the deoxynucleotide which is complementary to the base in the target position is incorporated into the growing complementary strand. When a deoxynucleotide is incorporated, inorganic pyrophosphate (PPi) is released. The released PPi is converted to adenosine-triphosphate (ATP) by ATP sulfurylase. Luciferase is used to convert the ATP to adenosine monophosphate (AMP), PPi and light. The luciferase reaction emits light at an intensity proportional to the concentration of ATP which is in turn dependent upon the amount of PPi produced and thus ultimately on the amount of deoxynucleotide incorporated. The light output may therefore be detected and correlated with the incorporation of the particular deoxynucleotide present at that time.

Where the target sequence contains repetitions of a particular base, increased amounts of the complementary deoxynucleotide will be incorporated, leading to a correspondingly increased emission of PPi which leads ultimately to an increased light intensity.

The reaction mixture also contains a nucleotide triphosphate degrading enzyme, apyrase. This enzyme degrades the excess remaining of the added deoxynucleotide and thereby circumvents the need for a wash cycle, that otherwise would be required to remove non-reacted deoxynucleotide between additions of the different deoxynucleotides. Apyrase also degrades the generated ATP and hence “turns off” the light from the reaction. Light emission reaches its maximum a few seconds after the addition of the deoxynucleotide, providing that it is complementary to the base in the next position of the template, and the enzymatic regeneration of the reaction is completed in approximately 60 seconds. Significant light is produced for approximately the first 30 seconds of the cycle and it is therefore desirable to follow the reaction for at least that period of time.

DNA sequencing performed according to the method described above is capable of generating high quality data in a simple fashion but the productivity of the method is not high if carried out as single reactions (typically 1 base read per 100 seconds).

From a first aspect the present invention provides an apparatus for simultaneously monitoring an array of reaction sites for light indicating that a reaction is taking place at a particular site, comprising an optically sensitive device arranged so that in use the light from a particular reaction site will impinge upon a particular predetermined region of said optically sensitive device, means for determining the light level impinging upon each of said predetermined regions and means to record the variation of said light level with time for each of said reaction sites.

Thus it will be seen that in accordance with the invention many potential reaction sites may be monitored at once with each site corresponding to a portion of the detection surface of the optically sensitive device. The optically sensitive device may then be scanned periodically in a predetermined sequence to give an electrical signal corresponding to the light level emitted at each of the sites. In general, the predetermined regions corresponding to respective reaction sites will be distinct, although this is not necessarily true in all cases. This allows many reactions to be run in parallel thereby improving the productivity of preferred methods such as the one described above. Moreover, it is possible to monitor the automatic repetition of reactions, e.g. with successive deoxynucleotides in a target base identification process in which apyrase is used to break down unreacted deoxynucleotides between reactions. This follows because it is not necessary to carry out a separate result collection step after each reaction.

Such an apparatus has clear advantages for use in identifying a target base in a DNA sequence. This may for example be in order to determine the unknown sequence of a DNA strand or to screen for single nucleotide polymorphisms. In both cases, a target base may be identified in many samples at once, thereby drastically reducing the time taken to carry out the process for a given number of samples.

When viewed from a second aspect therefore, the invention provides an apparatus for identifying a target base in a DNA sequence comprising a plate having a plurality of reaction sites, an optically sensitive device arranged so that in use light from respective reaction sites signifying the incorporation of a nucleotide will impinge upon separate detection portions of said optically sensitive device, means for determining the level of light impinging upon said separate detection portions, thereby indicating the level of light emitted from each reaction site, and means for recording the variation of light output from each of said reaction sites with time.

It will also be appreciated that the invention extends to a method of identifying a target base in a DNA sequence, comprising detecting the light level emitted from a plurality of reaction sites on respective portions of an optically sensitive device, converting the light impinging upon each of said portions of said optically sensitive device into an electrical signal which is distinguishable from the signals from all of said other regions, determining a light intensity for each of said discrete regions from the corresponding electrical signal, and recording the variations of said electrical signals with time.

Thus in accordance with the invention the progress of a plurality of light-emitting reactions may be monitored and recorded in real time. This enables a target base to be identified and thus is of particular benefit when used in the method disclosed in WO 98/13523, where deoxynucleotides may be added sequentially to a large number of reaction sites containing the target DNA and each can be monitored for the emission of light by the luciferase reaction while reagents are added to the remainder. This can significantly increase the efficiency of such a method.

It will be appreciated by those skilled in the art that the present invention is applicable both to the identification of a single target base in a DNA sequence e.g. when testing for a single base polymorphism and to the multiple repetition of such a method in order to sequence the target DNA.

The optically sensitive device may comprise an array of optical transducers—e.g. with each transducer corresponding to a subset of the reaction sites or even with an optical transducer corresponding to each reaction site. Preferably however the optically sensitive device comprises a single optical transducer. This is particularly advantageous in minimising the complexity of the optically sensitive device, and enabling a more compact design.

The reaction sites may be monitored from above, but preferably the reaction sites are monitored from underneath, with the parts of e.g. a plate beneath the reaction sites being at least partially transparent.

The reaction sites may simply be ‘spots’ of reagents on a flat plate which rely on surface tension. Preferably however the reaction sites are provided by wells in a reaction plate—e.g. a micro titre plate (MTP). In a particularly preferred embodiment many reactions are run in parallel in an MTP. After having added a small amount of the first deoxynucleotide to the sample in the first well of the MTP, the time required to complete the cycle to the next addition for this well (reading the signal and degrading the excess of deoxynucleotide) may be used to successively make addition of deoxynucleotides to the other samples of the MTP. Such an arrangement improves productivity, for example by two orders of magnitude (ie. one base read per second rather than one per 100 seconds), but it also calls for a detection system capable of continuously reading the light intensity from a plurality of reactions.

The plate may simply be suspended or supported on a surface, which is transparent or semi-transparent where the reactions are monitored from below. Preferably however the plate is in contact with heat regulating means in order to maintain the plate at a substantially constant and uniform temperature.

In particularly convenient arrangements masking means are provided between the reaction sites to help to avoid cross-contamination of light between the reaction sites which can occur, particularly when the reaction sites are provided by wells.

This is novel and inventive in its own right and thus from a yet further aspect the present invention provides a reaction medium comprising a plurality of reaction sites which are partially transparent at a lower part thereof, and opaque masking means between the reaction sites, said masking means being arranged so as to reduce the transmission of light between neighbouring reaction sites.

The masking means may comprise an opaque coating or the like applied selectively to the outer surfaces of said reaction sites so as to leave the lower parts thereof transparent or indeed the reaction sites may be made from two different materials, one of which is opaque. Preferably however the masking means are provided by channels in a block—for example a temperature regulating block. The channels can advantageously serve to receive reaction sites in the form of wells. Channels in a block may also be useful as masking means where the reaction sites are on a substantially flat plate rather than being an array of wells.

Most preferably the channels flare outwardly towards the lower part thereof in order to maximise the angles through which light may be emitted from the reaction sites or wells. It is also preferred that the masking means are at least partially reflective. Thus light which is initially emitted from reactions in a direction away from the optical path to the optically sensitive device, can be redirected towards the optically sensitive device.

In some embodiments of the invention light emitted from the reaction sites may impinge directly upon the optically sensitive device. In presently preferred embodiments however, optical means are provided between the reaction sites and the optically sensitive device to direct light from respective reaction sites onto respective detection portions of the optically sensitive device. Advantageously the optical means allows the optically sensitive device to be disposed remotely from the reaction sites. Said optical means may for example comprise a plurality of optical fibres—e.g. one per reaction site to direct light onto the appropriate portion of the optically sensitive device. Such an arrangement has the advantage in that it allows a great flexibility in the placement of the optically sensitive device since a line of sight is not required.

In preferred embodiments the optical means comprises an array of lenses. Most preferably there is a lens for each reaction site to be monitored and the array has a layout substantially similar to the layout of the reaction sites being monitored. An array of lenses is a relatively inexpensive way to enhance the intensity of light impinging upon the optically sensitive device. Such an array can also minimise cross-contamination of light from adjacent reaction sites and thus improve the resolution of the system. The array of lenses may be arranged in exact correspondence with the array of reaction sites, i.e. with each lens being spaced from its neighbours by the same amount as the corresponding reaction site. More preferably however the centre-to-centre spacing of the lenses of such an array is smaller than the corresponding centre-to-centre spacing of the reaction sites. This is beneficial in affording a greater efficiency in the collection of light from the reaction sites at the periphery of the array since light from these sights must be slightly angled in order to focus the image of the lens array onto a light-sensitive device which is smaller than the lens array itself.

Any optically sensitive device capable of resolving the part of its sensitive surface upon which light impinges may be used, although preferably the optically sensitive device comprises a charge-coupled device (CCD). A CCD has a matrix of electrical potential wells, each of which represents a pixel. Light impinging upon these pixels is converted into an electric charge. An optical or mechanical shutter may be used to enable the charge at each pixel to be read for each frame. However these add complexity to the apparatus and in the case of a mechanical shutter frequent repetitions for prolonged periods will means a relatively short lifetime or expensive manufacture. Preferably therefore a frame transfer CCD is used in which the charge at each pixel is stored in the respective electrical potential wells until a clocking signal moves the charge into corresponding non-light-sensitive storage areas for subsequent sequential reading. A CCD is particularly preferred since it allows a relatively high light sensitivity together with a relatively high resolution so as to enable a large number of reaction sites to be monitored at relatively low cost.

The rate at which the optically sensitive device is read—ie. the sampling rate—is preferably such that the time between consecutive reads is less than or equal to the time between the addition of reagents to consecutive reaction sites, where applicable. This ensures the correct monitoring of a plurality of reactions which are “triggered” at different times—e.g. by the addition of deoxynucleotides. Most preferably the sampling rate is sufficiently high to enable an evaluation of the kinetics of the reaction being monitored—e.g. the rate of increase or decrease in light output, the total light energy given out (i.e. the area under the graph of light intensity against time) and the like. This is beneficial since in certain reactions such information is useful because it acts as an indicator of the quality of the reaction. In certain convenient arrangements, where the invention is used in DNA sequencing, it is preferred that a measure of the total light energy output by a given reaction is determined in addition to or instead of the maximum level of said light. This has been found to give a better indication of the number of bases incorporated than the maximum level or maximum level alone.

Preferably the electrical signals are converted into a digital signal prior to calculating the corresponding light intensity. Digital conversion offers the advantages of easy manipulation e.g. by a personal computer (PC) or dedicated hardware such as a digital signal processor (DSP).

The charge transferred from each pixel may be individually converted into a digital value by a suitable A/D converter. Preferably however the charges from a block of neighbouring pixels e.g. 5 by 5 pixels are added together to produce an aggregate signal for that block, the aggregate signal being fed to an A/D converter. This method increases the signal-to-noise ratio of the converted digital signal as compared to that for the conversion and subsequent addition of individual pixels.

Each predetermined region or detection portion of the optically sensitive device may correspond to a single pixel. Preferably however each corresponds to a plurality of pixels, most preferably a large number e.g. several hundred pixels. All of the blocks of pixels corresponding to a particular reaction site may then be added together to give a light intensity for that site. This technique can be used with the present invention since only a relatively few areas of light need to be detected—e.g. 96 if a 96 well MTP is used as in the most preferred embodiment.

A preferred embodiment of the present invention will now be described, by way of example only, and with reference to the accompanying drawings in which:

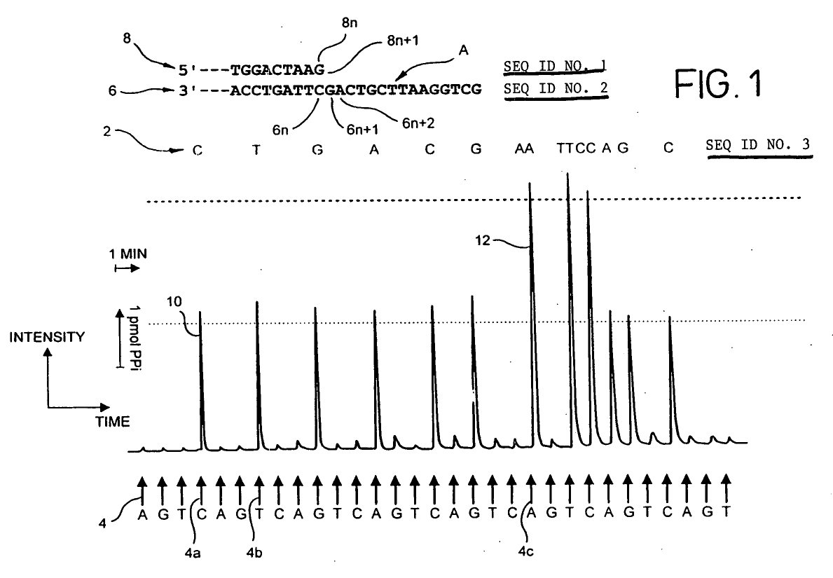

FIG. 1 is a graph of light intensity against time for a DNA sequence determination process which may be monitored in accordance with the present invention;

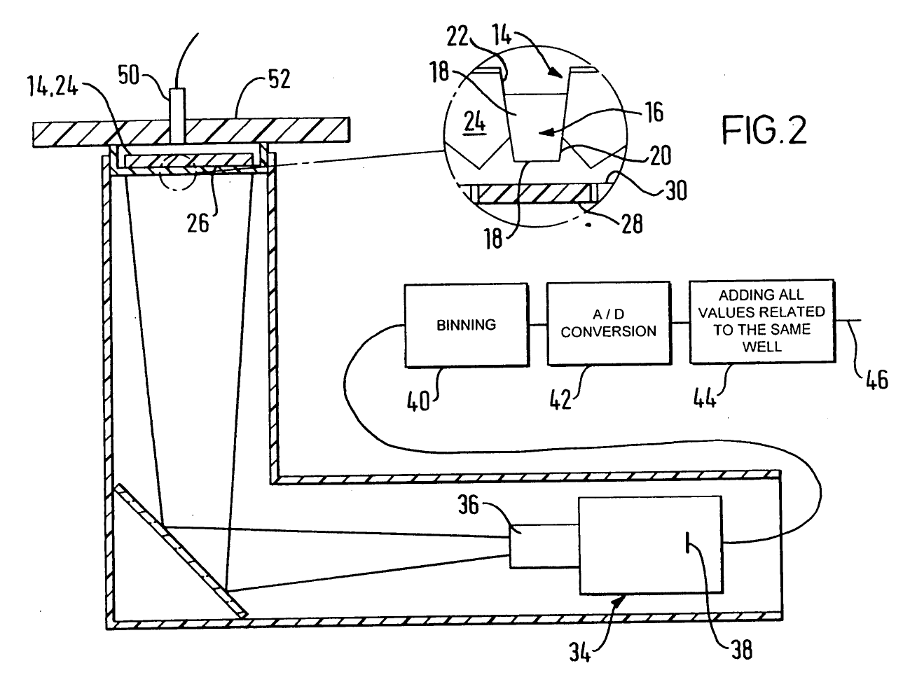

FIG. 2 is a schematic diagram of an embodiment of the present invention; and

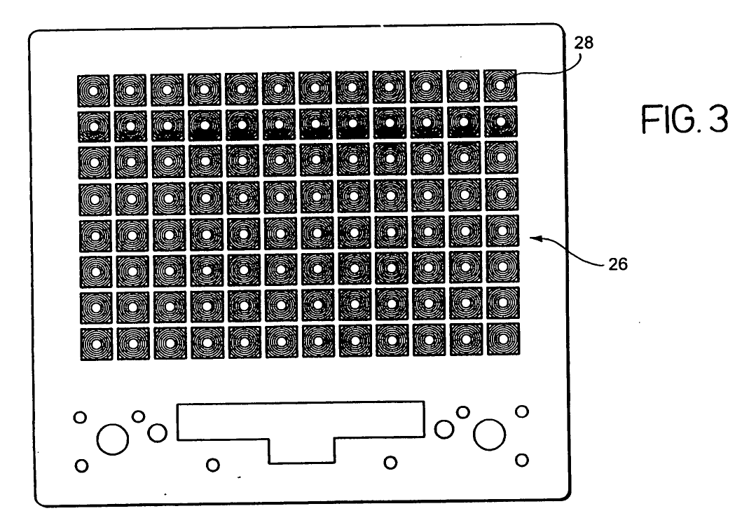

FIG. 3 is a more detailed view of the lens array used in the embodiment of FIG. 2.

Referring firstly to FIG. 1, a method of determining a DNA sequence 2 using the principle of sequencing-by-synthesis, will be briefly explained. A fuller explanation is given in WO 98/13523, although it is not essential for an understanding of the present invention.

A repeating series of adenine (A), guanine (G), thymine (T) and cytosine (C) deoxynucleotides are added at intervals of approximately one minute to the DNA fragment of interest which is a sequencing primer hybridized to a single stranded DNA fragment 6. A complementary strand 8 is successively built up in order to determine the sequence of the target 6. In the illustrated case the last base 8n of the complementary strand is a G. When A, G and T deoxynucleotides are successively added there is no significant reaction and therefore no significant light output. However when dCTP is added, the C nucleotide is incorporated since it complements the G base, 6n+1, which is the next in the target sequence. This incorporation is accompanied by a corresponding production of inorganic pyrophosphate which is converted into ATP by ATP sulfurylase which is already in the reaction mixture.

The ATP produced causes luciferase, also present, to emit light. This is shown on the graph by the left-most peak 10. This gives the first letter C in the determined sequence 2. The reaction mixture also contains a nucleotide triphosphate degrading enzyme, apyrase, that degrades the excess remaining of the added deoxynucleotide and thereby prepares the reaction mixture for the next cycle. Apyrase also degrades the generated ATP and hence “turns off” the light from the reaction. As may be seen, the cycle is repeated with the next nucleotide to be incorporated 4b being a T (to complement the A at 6n+2 in the target sequence).

It will be seen that when dATP is added at 4c, approximately twice as much light 12 is given off as compared to the previous nucleotide incorporations. This is explained by the fact that the target sequence contains a repetition of the T base at 14. To complement these, twice as much dATP is incorporated and thus the DNA polymerase reaction gives twice as much PPi.

It will be seen from the above that by constructing the complementary strand 8, the sequence 2 is determined, the complement of which gives the target sequence 6.

Turning now to FIGS. 2 and 3, an apparatus in accordance with a preferred embodiment of the invention is shown. The reactions of interest take place in the wells of a 96 well MTP 14, which may be seen more clearly in the enlarged fragment. The MTP 14 comprises an array of wells 16 in an 8×12 configuration which is moulded or vacuum formed from a suitable transparent plastics material. For convenience the whole MTP 14 is made from the same material although alternatively just the base 18 may be transparent. In the embodiment described, the thickness of the well walls 20 is approximately 0.3 mm.

The wells 16 are received in channels 22 in a heating block 24 which is made of aluminium so as to have a high reflectance for visible light. The walls of the channels 22 taper downwardly from the top although flare out at the bottom end 20 in order to avoid obscuring light emitted through the well.

The DNA samples to be analysed are placed in the respective wells 16 and the MTP is then located in the apparatus, where the reagents 48 are added by a dispenser 50 which is computer-controlled to deliver a precise volume of the required reagent from a reagent cassette (not shown). The dispenser 50 is moved across the MTP 14 by means of an x-y table 52. Alternatively the reagents may be pre-dispensed, e.g. manually, into the wells, before the MTP is placed in the apparatus.

Below the MTP 14 by a distance of 9 mm is a lens array 26. As may be seen from the detail view, the lens array 26 is arranged so that there is a separate lens 28 below each well 16 in the MTP. However the centre-to-centre spacings of the lenses 28 are all 8.75 mm, whereas the centre-to-centre spacings of the wells 16 are all 9.0 mm. This difference in the respective spacings of the wells 16 and lenses 28 emulates the effect of a field lens to reduce the difference in efficiency of light collection between the wells in the centre of the MTP and those at the periphery. The areas 30 between the lenses 28 are opaque and so will be detected as dark areas by the camera. The lens array is such that light coming down at any angle from reactions in the wells 16 will pass through the lens 28 or will be absorbed by the opaque area 30 rather than entering an adjacent lens. The possibility of cross-contamination of light between the wells 16 is thereby avoided.

Vertically below the lens array 26 is a mirror 32 inclined at approximately 45° to deflect light horizontally. Further along the optical path is a CCD camera 34. The camera has a lens 36 which focuses incoming light onto the CCD chip 38 inside the camera. The CCD chip 38 is a frame-transfer CCD chip and has 500×290 charge elements. Each of the charge elements of the CCD chip corresponds to a pixel and develops a charge when illuminated proportional to the intensity of the incident light. A clock signal of approximately 1 Hz is generated by a suitable oscillator in order to shift charges from the light sensitive elements to positions within the chip which are screened from light. During the interval between the main clock pulses, the charges of blocks of 5×5 pixels are added together in a process called binning which is carried out by ‘binning’ circuit 40. The aggregate values are then converted to a digital format by an analogue to digital convertor 42. A further analysis stage 44 correlates the digital signals for the blocks with corresponding wells 14 on the MTP and adds the values of all the blocks for a given well together. In the desired example in which each block is made up of 5×5 pixels there will be a potential total of 5800 blocks. As the MTP has 96 wells there is a potential maximum of 60 blocks per well. In practice some blocks will correspond to the gaps between the wells and each well will be associated with fewer blocks. A serial connection carries the data to a PC for recording and displaying the light intensity for each well. The results may be displayed in any convenient format. For example a graph such as the one shown in FIG. 1 may be displayed or be available for display for each well 16.

Although the data connection 46 to a PC is shown after the binning 40, A/D conversion 42 and addition 44 stages, alternative arrangements are possible. For example some or all of these stages may be performed within the PC. Further processing may also be performed in the PC e.g. a pre-screening designed only to display the light outputs corresponding nucleotide incorporations—i.e. to apply a threshold light level. Indeed this may be implemented at an earlier stage in the system such as the CCD or associated circuitry to record the light output only during an incorporation event when the level is above a predefined threshold.

It will be appreciated by those skilled in the art that whilst a process of determining an unknown DNA sequence has been described, the invention may be used equally for identifying single nucleotide polymorphisms for example.

1-17. (canceled)

18. A method of identifying a target base in the DNA sequences of a plurality of samples, comprising:
providing said plurality of samples at respective reaction sites;
dispensing at least one reagent into each of said samples;
detecting simultaneously the light level emitted from each of said reaction sites on respective portions of an optically sensitive device;
converting the light impinging upon each of said portions of said optically sensitive device into an electrical signal which is distinguishable from the signals from all of said other portions, wherein the interval between successive readings of the state of the optically sensitive device is less than the time between the addition of reagents to consecutive reaction sites;
determining a light intensity for each of said discrete portions from the corresponding electrical signal;
recording the variations of said electrical signals indicating light intensity with time;
determining and recording the times at which a series of peaks in light intensity occur; and
associating each of said peaks with the addition of a particular reagent to the corresponding sample.

19.-20. (canceled)


SEQUENCE LISTING (3 sequences)

SEQ ID NO: 1 - DNA, 9 bp, Artificial
  oligonucleotide
tggactaag

SEQ ID NO: 2 - DNA, 24 bp, Artificial
  oligonucleotide
acctgattcgactgcttaaggtcg

SEQ ID NO: 3 - DNA, 15 bp, Artificial
  oligonucleotide
ctgacgaattccagc